(19) United States
(12) Patent Application Publication
(10) Pub. No.: US 2014/0308733 A1
(43) Pub. Date: Oct. 16, 2014

(54) METHOD FOR INDUCING GERMINATION OF SPORE FORMING BACTERIUM

(71) Applicants: Toki Nishiyama, Kanagawa (JP); Nobuhisa Ashida, Kanagawa (JP); Gentaro Yasuda, Kanagawa (JP); Yukie Tadano, Kanagawa (JP)

(72) Inventors: Toki Nishiyama, Kanagawa (JP); Nobuhisa Ashida, Kanagawa (JP); Gentaro Yasuda, Kanagawa (JP); Yukie Tadano, Kanagawa (JP)

(73) Assignee: CALPIS CO., LTD.

(21) Appl. No.: 14/343,506

(22) PCT Filed: Sep. 6, 2012
(86) PCT No.: PCT/JP2012/072784

§ 371 Date: Jun. 23, 2014

(30) Foreign Application Priority Data
Sep. 8, 2011 (JP) 2011-196153

(51) Int. Cl. C12N 3/00

(52) U.S. Cl. CPC C12N 3/00
USPC 435/242; 562/478; 562/475; 562/576; 568/337; 435/252.1; 435/252.5

(57) ABSTRACT

The present application relates to a method for inducing germination of a spore forming bacterium, specifically, a method for inducing germination of a spore forming bacterium, comprising: using one or more compounds having a phenolic backbone to induce germination of a spore forming bacterium selected from the group consisting of the genus Bacillus, the genus Geobacillus, and the genus Aeribacillus; to a germination inducer for a spore forming bacterium; and to a kit for use in germination of a spore forming bacterium.

### TECHNICAL FIELD

The present invention relates to a method for inducing germination of a spore forming bacterium such as a bacterium of the genus Bacillus, an inducer, and a kit therefor.

### BACKGROUND ART

Since spore forming bacteria such as bacteria of the genus Bacillus have environmental resistance, such as heat resistance, desiccation resistance, plant disease-and-pest controlling ability in a state of spores, as well as ability for regulating the functions of intestines in domestic animals and fowls, fish, etc., especially applications in the agricultural field have been envisaged (Patent Literature 1). For example, it has been reported that, by adding bacteria of the genus Bacillus together with unfermented organic substances to soil, a soil disease caused by plant pathogenic bacteria of the genus Streptomyces can be controlled (Patent Literature 2). Further, it has been known that bacteria of the genus Bacillus contribute to composting of organic wastes, moreover it has been proposed that bacteria of the genus Geobacillus should be mixed with a natural thickening polysaccharides and a water absorbing fibrous material and used as a composting conditioner for adjusting appropriately the hardness of a compost feedstock (Patent Literature 3).

Although spore forming bacteria maintain a dormant state for a long term in a form of spores, once germination being induced, the bacteria become vegetative cells and proliferate. With respect to a method for promoting such germination, for example, Patent Literature 4 discloses a spore germination culture medium, which enhances a spore germination rate, containing amino acids such as Ala, Val, Asn, and Gln, as germination factors for Bacillus subtilis. Further, Patent Literature 5 discloses a composition of an agri-horticultural antimicrobial agent containing spores of Bacillus subtilis and a spore germination promotion agent therefor. Patent Literature 6 discloses a method for treating an organic wastewater, in which a sludge containing bacteria of the genus Bacillus and a germination promotion agent are mixed.

Further, with respect to an application of compounds having a phenolic backbone related to the present invention, Patent Literature 7 describes a use of compounds having a methoxyphenolic backbone (vanillin, vanillic acid, and ferulic acid) in a method for detecting a guaiacol producing microorganism. Further, a Non Patent Literature 1 describes utilization of vanillin, vanillic acid and ferulic acid in growing Bacillus subtilis.

### PRIOR ART LITERATURE

### Patent Literature

Patent Literature 1: Japanese Patent Publication (Kokai) No. 2007-236286 A
Patent Literature 2: Japanese Patent Publication (Kokai) No. 2007-153873 A
Patent Literature 3: Japanese Patent No. 4272251
Patent Literature 4: Japanese Patent Publication (Kohyo) No. 2001-511356 A
Patent Literature 5: Japanese Patent Publication (Kokai) No. 8-175921 A (1996)
Patent Literature 6: Japanese Patent Publication (Kokai) No. 2001-286884 A
Patent Literature 7: Japanese Patent Publication (Kokai) No. 2011-19506 A

### Non Patent Literature

Non Patent Literature 1: G. Gurujeyalakshmi and A. Mahadevan, Current Microbiology, 1987; 16: 69-73

### SUMMARY OF INVENTION

### Problem to be Solved by the Invention

An object of the present invention is to provide a method for inducing germination of a spore forming bacterium, an agent and a kit therefor.

### Means for Solving the Problem

The inventors have now found that a compound having a phenolic backbone induces germination of a spore forming bacterium and have completed the present invention based thereon.

The present invention includes the following characteristics.

According to the first aspect, the present invention provides a method for inducing germination of a spore forming bacterium, comprising: using one or more compounds having a phenolic backbone to induce germination of a spore forming bacterium selected from the group consisting of the genus Bacillus, the genus Geobacillus, and the genus Aeribacillus.

According to an embodiment thereof, the method is characterized in that the compound is vanillin, vanillic acid, ferulic acid, or coumaric acid, or a salt thereof.

According to another embodiment, the method is characterized in that the spore forming bacterium is Bacillus subtilis, Bacillus licheniformis, Bacillus amyloliquefaciens, or Bacillus coagulans, Geobacillus stearothermophilus, or Aeribacillus pallidus.

According to further embodiment, the effective concentration of the compound is 1 to 4000 ppm.

According to the second aspect, the present invention further provides a germination inducer, which is for a spore forming bacterium belonging to the genus Bacillus, the genus Geobacillus, or the genus Aeribacillus, comprising one or more compounds having a phenolic backbone.

According to an embodiment thereof, the germination inducer is characterized in that the compound is vanillin, vanillic acid, ferulic acid, or coumaric acid, or a salt thereof.

According to another embodiment, the germination inducer is characterized in that the spore forming bacterium is Bacillus subtilis, Bacillus licheniformis, Bacillus amyloliquefaciens, or Bacillus coagulans, Geobacillus stearothermophilus, or Aeribacillus pallidus.

According to the third aspect, the present invention further provides a kit for use in germination of a spore forming bacterium, comprising: a bacterial cell selected from the group consisting of the genus Bacillus, the genus Geobacillus, and the genus Aeribacillus, wherein the bacterial cell has formed a spore; and/or the spore thereof; and the germination inducer.

According to an embodiment thereof, the kit is characterized in that the spore forming bacterium is Bacillus subtilis, Bacillus licheniformis, Bacillus amyloliquefaciens, or Bacillus coagulans, Geobacillus stearothermophilus, or Aeribacillus pallidus.

According to another embodiment, the kit is characterized in that the spore forming bacterium is Bacillus subtilis C-3102 (FERM BP-1096), Bacillus subtilis NBRC 12195, Bacillus subtilis NBRC 14206, Bacillus licheniformis NBRC 12200, Bacillus subtilis sp spizizenii NBRC 101239, Bacillus amyloliquefaciens NBRC 3022, Bacillus amyloliquefaciens NBRC 3032, Bacillus coagulans NBRC 12583, Bacillus coagulans NBRC 3887, Geobacillus stearothermophilus NBRC 13737, or Aeribacillus pallidus TK 6004 (the same strain as Bacillus pallidus TK6004 (accession number FERM BP-08597)).

The present specification includes the contents of the specification and/or drawings described in Japanese Patent Application No. 2011-196153, to which the present application claims priority.

### Effect of the Invention

Since germination of a spore forming bacterium of interest can be induced according to the present invention irrespective of any nutrient component, the bacterium changed into a vegetative form can proliferate rapidly by obtaining necessary nutrient components thereafter, and the invention will therefore be advantageous in promoting composting or producing a spore forming bacterium. Moreover, since a nutrient component is not added, the invention can bring an advantage that disinfection can be carried out efficiently by inducing solely germination of harmful spore forming bacteria contained in litter or a food.

### BRIEF DESCRIPTION OF THE DRAWINGS

FIG. 1-1 This figure is a graph showing changes of OD 630 values over time, after Bacillus subtilis C-3102 (FERM BP-1096) was cultured: in a sporulation medium for 3 days, then the bacterial cells were suspended in distilled water containing ferulic acid or vanillic acid as the germination inducer according to the present invention; in distilled water containing L-alanine (“L-Ala”) as a control; or in distilled water (“D. W.”) as a negative control, and subsequently shaken at 37° C. and 150 rpm. When germination is induced, the OD 630 value decreases.

FIG. 1-2 This figure is a graph showing changes of OD 630 values over time, after Bacillus licheniformis NBRC 12200 was cultured: in a sporulation medium for 3 days, then the bacterial cells were suspended in distilled water containing ferulic acid or vanillic acid as the germination inducer according to the present invention; in distilled water containing L-alanine (“L-Ala”) as a control; or in distilled water (“D. W.”) as a negative control, and subsequently shaken at 37° C. and 150 rpm.

FIG. 1-3 This figure is a graph showing changes of OD 630 values over time, after Bacillus subtilis sp spizizenii NBRC 101239 was cultured: in a sporulation medium for 3 days, then the bacterial cells were suspended in distilled water containing ferulic acid or vanillic acid as the germination inducer according to the present invention; in distilled water containing L-alanine (“L-Ala”) as a control; or in distilled water (“D. W.”) as a negative control, and shaken at 37° C. and 150 rpm.

FIG. 1-4 This figure is a graph showing changes of OD 630 values over time, after Geobacillus stearothermophilus NBRC 13737 was cultured: in a sporulation medium for 3 days, then the bacterial cells were suspended in distilled water containing ferulic acid or vanillic acid as the germination inducer according to the present invention; in distilled water containing L-alanine (“L-Ala”) as a control; or in distilled water (“D. W.”) as a negative control, and subsequently shaken at 37° C. and 150 rpm.

FIG. 1-5 This figure is a graph showing changes of OD 630 values over time, after Aeribacillus pallidus TK 6004 (which is the same strain as Bacillus pallidus TK6004 (FERM BP-08597)) was cultured: in a sporulation medium for 3 days, then the bacterial cells were suspended in distilled water containing ferulic acid or vanillic acid as the germination inducer according to the present invention; in distilled water containing L-alanine (“L-Ala”) as a control, or in distilled water (“D. W.”) as a negative control; and shaken at 37° C. and 150 rpm.

FIG. 1-6 This figure is a graph showing changes of OD 630 values over time, after Bacillus licheniformis NBRC 12195 was cultured: in a sporulation medium for 3 days, then the bacterial cells were suspended in distilled water containing ferulic acid or vanillic acid as the germination inducer according to the present invention; in distilled water containing L-alanine (“L-Ala”) as a control, or in distilled water (“D. W.”) as a negative control; and subsequently shaken at 37° C. and 150 rpm.

FIG. 1-7 This figure is a graph showing changes of OD 630 values over time, after Bacillus licheniformis NBRC 14206 was cultured in a sporulation medium for 3 days, then the bacterial cells were suspended in distilled water containing ferulic acid or vanillic acid as the germination inducer according to the present invention; in distilled water containing L-alanine (“L-Ala”) as a control; or in distilled water (“D. W.”) as a negative control, and shaken at 37° C. and 150 rpm.

FIG. 1-8 This figure is a graph showing changes of OD 630 values over time, after Bacillus amyloliquefaciens NBRC 3022 was cultured: in a sporulation medium for 3 days, then the bacterial cells were suspended in distilled water containing ferulic acid or vanillic acid as the germination inducer according to the present invention; in distilled water containing L-alanine (“L-Ala”) as a control, or in distilled water (“D. W.”) as a negative control; and subsequently shaken at 37° C. and 150 rpm.

FIG. 1-9 This figure is a graph showing changes of OD 630 values over time, after Bacillus amyloliquefaciens NBRC 3032 was cultured: in a sporulation medium for 3 days, then the bacterial cells were suspended in distilled water containing ferulic acid or vanillic acid as the germination inducer according to the present invention; in distilled water containing L-alanine (“L-Ala”) as a control; or in distilled water (“D. W.”) as a negative control; and subsequently shaken at 37° C. and 150 rpm.

FIG. 1-10 This figure is a graph showing changes of OD 630 values over time, after Bacillus coagulans NBRC 12583 was cultured: in a sporulation medium for 3 days, then the bacterial cells were suspended in distilled water containing ferulic acid or vanillic acid as the germination inducer according to the present invention; in distilled water containing L-alanine (“L-Ala”) as a control, or in distilled water (“D. W.”) as a negative control; and subsequently shaken at 37° C. and 150 rpm.

FIG. 1-11 This figure is a graph showing changes of OD 630 values over time, after Bacillus coagulans NBRC 3887 was cultured: in a sporulation medium for 3 days, then the bacterial cells were suspended in distilled water containing ferulic acid or vanillic acid as the germination inducer according to the present invention, in distilled water containing L-alanine (“L-Ala”) as a control; or in distilled water (“D. W.”) as a negative control; and subsequently shaken at 37° C. and 150 rpm.

FIG. 2-1 This figure is a graph showing the influence of the concentration of ferulic acid on germination of Bacillus subtilis C-3102 (FERM BP-1096).

FIG. 2-2 This figure is a graph showing the influence of the concentration of vanillic acid on germination of Bacillus subtilis C-3102 (FERM BP-1096).

FIG. 3 This figure is a graph showing changes of OD 630 values over time, after Bacillus subtilis C-3102 (FERM BP-1096) was cultured: in a sporulation medium for 3 days, then the bacterial cells were suspended in distilled water containing ferulic acid, vanillic acid, or p-coumaric acid as the germination inducer according to the present invention; or in distilled water (“D. W.”) as a negative control; and subsequently shaken at 37° C. and 150 rpm.

### MODE FOR CARRYING OUT THE INVENTION

The present invention will be described in more detail.

### <Method for Inducing Germination>

The present invention provides a method for inducing germination of a spore forming bacterium, comprising: using one or more compounds having a phenolic backbone to induce germination of a spore forming bacterium selected from the group consisting of the genus Bacillus, the genus Geobacillus, and the genus Aeribacillus.

The compounds having a phenolic backbone include compounds having a methoxyphenolic backbone. For example, such compound is a compound represented by the following formula, or a salt thereof:

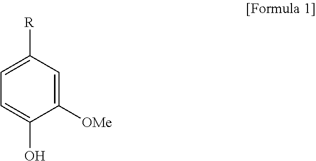

wherein, R is —H, —OH, —C(O)H, —C(O)CH₃, —COOH, a C1-C3 alkyl group, or a C1-C3 alkenyl group, wherein the alkyl group and alkenyl group may be substituted with —OH, —C(O)H or COOH. The compound is also described in Japanese Patent Publication (Kokai) No. 2011-19506 A from the present applicant.

Any compound may be included in the compounds insofar as germination of a spore forming bacterium can be induced, examples thereof include, but not limited to, vanillin (R: —C(O)H), vanillic acid (R: —COOH), ferulic acid (R: —CH═CH—COOH), guaiacol (R: —H), 4-hydroxy-3-methoxyphenylacrylic aldehyde (R: —CH═CH—C(O)H), 4-hydroxy-3-methoxyphenylpropionic acid (R: —CH₂CH₂COOH), 4-hydroxy-3-methoxyphenylmethyl alcohol (R: —CH₂OH), methoxyhydroquinone (R: —OH), 4-hydroxy-3-methoxyphenylacetaldehyde (R: —C(O)CH₃), and 4-vinyl guaiacol (R: —CH₂═CH₂), and a salt thereof. All of the compounds are metabolic products to be produced by a microorganism. Preferable compounds are vanillin, vanillic acid, ferulic acid, and a salt thereof.

Alternatively, the compound having a phenolic backbone may be hydroxycinnamic acid, for example, o-, m-, or p-coumaric acid, or a salt thereof.

Examples of a salt of the compound include salts of alkali metals, such as Na and K, and ammonium salts with ammonia or any of organic amines. Examples of the organic amines include aliphatic amines and aromatic amines.

The spore forming bacterium is selected from the group consisting of the genus Bacillus, the genus Geobacillus, and the genus Aeribacillus.

Examples of bacteria belonging to the genus Bacillus include, but not limited to, Bacillus subtilis, Bacillus amyloliquefaciens, Bacillus pumilus, Bacillus lentus, Bacillus laterosporus, Bacillus alvei, Bacillus popilliae, Bacillus licheniformis, Bacillus coagulans, Bacillus cereus, Bacillus halodurans, Bacillus subtilis natto, Bacillus acidicola, Bacillus acidopullulyticus, Bacillus acidovorans, Bacillus aeolius, Bacillus aestuarii, Bacillus agaradhaerens, Bacillus akibai, Bacillus alcaliinulinus, Bacillus alcalophilus, Bacillus algicola, Bacillus alkalitolerans, Bacillus alkalogaya, Bacillus alveayuensis, Bacillus amiliensis, Bacillus aminovorans, Bacillus aquimaris, Bacillus arbutinivorans, Bacillus arenosi, Bacillus arseniciselenatis, Bacillus arsenicus, Bacillus arvi, Bacillus asahii, Bacillus atrophaeus, Bacillus axarquiensis, Bacillus azotoformans, Bacillus badius, Bacillus baekryungensis, Bacillus barbaricus, Bacillus bataviensis, Bacillus benzoevorans, Bacillus bogoriensis, Bacillus borophilicus, Bacillus borotolerans, Bacillus caldolyticus, Bacillus caldotenax, Bacillus caldovelox, Bacillus carboniphilus, Bacillus casamancensis, Bacillus catenulatus, Bacillus cellulosilyticus, Bacillus sphaericus, Bacillus thuringiensis, and Bacillus clausii.

Examples of bacteria belonging to the genus Geobacillus include, but not limited to, Geobacillus anatolicus, Geobacillus kaue, Geobacillus caldoproteolyticus, Geobacillus caldoxylosilyticus, Geobacillus debilis, Geobacillus gargensis, Geobacillus kaustophilus, Geobacillus stearothermophilus, Geobacillus thermocatenulatus, Geobacillus thermodenitrificans, Geobacillus thermoglucosidasius, Geobacillus thermoleovorans, Geobacillus uralicus, Geobacillus uzenensis, and Geobacillus vulcani.

Examples of bacteria belonging to the genus Aeribacillus include, but not limited to, Aeribacillus pallidus (International Journal of Systematic and Evolutionary Microbiology, (2010), 60, 1600-1604).

The bacteria listed above as examples are publicly known as spore forming bacteria (Japanese Patent Publication (Kokai) No. 2007-236286 A, Japanese Patent Publication (Kokai) No. 2007-332128 A, etc.).

In Examples described below, Bacillus subtilis, Bacillus licheniformis, Bacillus amyloliquefaciens, Bacillus coagulans, Geobacillus stearothermophilus, and Aeribacillus pallidus; specifically, Bacillus subtilis C-3102 (FERM BP-1096), Bacillus subtilis NBRC 12195, Bacillus subtilis NBRC 14206, Bacillus licheniformis NBRC 12200, Bacillus subtilis sp spizizenii NBRC 101239, Bacillus amyloliquefaciens NBRC 3022, Bacillus amyloliquefaciens NBRC 3032, Bacillus coagulans NBRC 12583, Bacillus coagulans NBRC 3887, Geobacillus stearothermophilus NBRC 13737, or Aeribacillus pallidus TK6004 (FERM BP-08597) was selected and each of the strains was examined for the formation of spores and the germination.

In the culture to form spores from the spore forming bacterium, a nitrogen source and a carbon source that a bacterium of the genus Bacillus, a bacterium of the genus Geobacillus, or a bacterium of the genus Aeribacillus can utilize, may be preferably used. Examples of the nitrogen source include proteins, such as meat extracts, yeast extracts, and peptone, inorganic nitrogen sources, such as nitrate and ammonium salts, amino acids, peptides, and corn steep liquors. Examples of the carbon source include saccharides, such as glucose, sucrose, lactose, galactose, and maltose, starches, amino acids, peptides, and proteins. Although an optimal pH of a culture medium should be selected according to the type of the bacterium, it is, for example, in a range of pH 7.0 to 9.0.

The culture temperature is ordinarily approx. 25 to 40° C., but may exceed 40° C. if the bacterium is heat resistant.

The culture may be conducted ordinarily under aerobic conditions by a batch culture, a stirring culture, or a shaking culture.

Bacterial cells, which were spore-formed by the above-mentioned procedures, are filtrated, separated by a separating means such as centrifugation, and charged into a culture medium for germination to induce germination. Although a culture medium for germination may be similar to the sporulation medium, one or more compounds having a phenolic backbone are added to the culture medium for germination. Preferable examples of the compounds include compounds having a methoxyphenolic backbone, such as vanillin, vanillic acid, or ferulic acid, or a salt thereof. Other examples of compounds having a phenolic backbone include hydroxycinnamic acid, such as o-, or p-coumaric acid, preferably p-coumaric acid, or a salt thereof. With respect to the effective concentration of a compound having a phenolic backbone in a culture medium, any concentration may be used insofar as germination is induced, however, in consideration of the solubility of the compounds and negative influence on growth of microorganisms, for example, it is 1 to 4000 ppm, or 5 to 3000 ppm; preferably 10 to 2000 ppm, or 10 to 1000 ppm, or 10 to 600 ppm; and further preferably 100 to 600 ppm. Specifically, for ferulic acid a concentration range of 1 to 3900 ppm, or 5 to 3000 ppm; preferably 10 to 2000 ppm, 0 to 1000 ppm, or 10 to 600 ppm; and further preferably 100 to 600 ppm; for vanillin a concentration range of 1 to 3050 ppm, or 5 to 3000 ppm; preferably 10 to 2000 ppm, or 10 to 1000 ppm, or 10 to 600 ppm; and for vanillic acid a concentration range of 1 to 3400 ppm, or 5 to 3000 ppm; preferably 10 to 2000 ppm e.g. 10 to 1800 ppm, or 10 to 1000 ppm, or 10 to 600 ppm; and further preferably 100 to 600 ppm, may be used. For coumaric acid a similar concentration range, namely 1 to 4000 ppm, or 5 to 3000 ppm; preferably 10 to 2000 ppm, or 10 to 1000 ppm, or 10 to 600 ppm; and further preferably 100 to 600 ppm, is effective.

Since it is said that existence of a carbon source such as glucose and an amino acid such as L-Ala is generally favorable for germination, it is conceivable that germination can be induced in any of culture media for germination, such as a sporulation medium (a nutrient culture medium), and TS culture medium, with the composition containing a proper amount of a carbon source. Further, when the spore formation rate is poor, it becomes difficult to examine the germination induction effect, and therefore spore formation is better to be confirmed before a germination test by culturing after Gram staining, and a heat shock treatment (65° C., 35 min).

A spore forming bacterium, in which germination can be induced by vanillin, vanillic acid, ferulic acid, or coumaric acid, or a salt thereof, can be selected from spore forming bacteria belonging to the genus Bacillus, the genus Geobacillus, or the genus Aeribacillus, for example, from the above-listed bacterial species. Especially preferable bacterial species are Bacillus subtilis, Bacillus licheniformis, Bacillus amyloliquefaciens, Bacillus coagulans, Geobacillus stearothermophilus, and Aeribacillus pallidus, and further specifically, but not limited to, Bacillus subtilis C-3102 (FERM BP-1096), Bacillus subtilis NBRC 12195, Bacillus subtilis NBRC 14206, Bacillus licheniformis NBRC 12200, Bacillus subtilis subspecies spizizenii NBRC 101239, Bacillus amyloliquefaciens NBRC 3022, Bacillus amyloliquefaciens NBRC 3032, Bacillus coagulans NBRC 12583, Bacillus coagulans NBRC 3887, Geobacillus stearothermophilus NBRC 13737, or Aeribacillus pallidus TK6004 (FERM BP-08597).

It is possible to determine whether spores have germinated, after measuring an OD 630 value (turbidity at 630 nm) of the liquid culture, based on decrease in the value (Literature: Microbiology, (1950), 4, 3, 330-338). Briefly, bacterial cells after culturing are washed with distilled water and diluted with distilled water to OD 630=1.0, which is then mixed with an equal volume of a germination induction solution, and the turbidity at 630 nm is measured by a UV spectrophotometer at room temperature.

### <Germination Inducer>

The present invention further provides a germination inducer, which is for a spore forming bacterium belonging to the genus Bacillus, the genus Geobacillus, or the genus Aeribacillus, comprising one or more compounds having a phenolic backbone.

Preferable examples of the compounds are as described above vanillin, vanillic acid, ferulic acid, or coumaric acid, or a salt thereof.

A germination inducer may be either in a solid form or in a liquid form. In the case of a solid form, the compound may be used as such, or mixed with an agriculturally acceptable carrier or excipient to be formulated into any form, such as powder, pellet, and granule. Meanwhile, in the case of a liquid form, a germination inducer may take a form dissolved, suspended, or emulsified in a solvent, such as water or organic solvent (e.g. EtOH, DMSO, acetone, or acetonitrile), or optionally in a concentrated form, which can be diluted just before use. A germination inducer may contain according to need an agriculturally acceptable additive, such as a surfactant, a suspending agent, a coloring agent, and a stabilizer.

Although there is no particular restriction on the concentration of the compound having a phenolic backbone in a germination inducer, it is, for example, approximately 1 to 100% by weight.

The concentration of the compound at the time of inducing germination is adjusted to, for example, 1 to 4000 ppm, or 5 to 3000 ppm; preferably 10 to 2000 ppm, or 10 to 1000 ppm, or 10 to 600 ppm; and further preferably 100 to 600 ppm.

### <Kit>

The present invention provides further a kit comprising a bacterial cell selected from the group consisting of the genus Bacillus, the genus Geobacillus, and the genus Aeribacillus, wherein the bacterial cell has formed spores; and/or the spores thereof; and the germination inducer comprising one or more compounds having a phenolic backbone.

The bacterial cell that has formed a spore can be obtained by the culture for forming spores. A spore forming bacterium to be included in the kit is preferably a bacterium applicable as a useful substance, such as a soil improving agent, a composting promotion agent, and a sludge treatment agent, and examples thereof include bacterial species of Bacillus subtilis, Bacillus licheniformis, Bacillus amyloliquefaciens, Bacillus coagulans, Geobacillus stearothermophilus, and Aeribacillus pallidus.

Examples of the bacterial species include, but not limited to, Bacillus subtilis C-3102 (FERM BP-1096), Bacillus subtilis NBRC 12195, Bacillus subtilis NBRC 14206, Bacillus licheniformis NBRC 12200, Bacillus subtilis sp spizizenii NBRC 101239, Bacillus amyloliquefaciens NBRC 3022, Bacillus amyloliquefaciens NBRC 3032, Bacillus coagulans NBRC 12583, Bacillus coagulans NBRC 3887, Geobacillus stearothermophilus NBRC 13737, or Aeribacillus pallidus TK6004 (FERM BP-08597). Especially preferable bacteria are bacteria belonging to the genus Bacillus, and more preferably Bacillus subtilis, such as Bacillus subtilis C-3102, and Bacillus amyloliquefaciens NBRC 3022, or bacteria belonging to the genus Aeribacillus, and more preferably Aeribacillus pallidus, such as Aeribacillus pallidus TK6004 (FERM BP-08597).

The bacterial cells having formed spores and/or the spores thereof are preferably in a dry form dried by a drying means such as freeze-drying, but may be also in a wet form.

The bacterial cells and/or the spores and the germination inducer included in the kit may be mixed and then administered to a liquid or solid substance to be treated (e.g. a culture medium). If they are added simultaneously, the bacterium of interest is easily manageable. However, there is no particular rule concerning the order of addition of a spore forming bacterium and a germination inducer to soil, litter, sludge, etc., and the germination inducer may be used on a spore forming bacterium contained in the objective substance, or on a spore forming bacterium added in advance. After the administration, by keeping the culture conditions properly, spores germinate and the bacterial cells change to a vegetative form, which can then proliferate.

By utilizing the method for inducing germination, the agent, and the kit according to the present invention, spore forming bacteria which may cause pollution of a beverage or a food can be disinfected after germinating the same. Further, in using litter (rice straw, sawdust, chaff, etc.), by a treatment with heat or a disinfectant after germination of spore forming bacteria causing pollution, disinfection can be easily performed. Further, with respect to a domestic waste or the like containing microorganisms causing a bad smell, proliferation of the microorganisms causing the bad smell can be suppressed by adding a combination of a spore forming bacterium and the germination inducer according to the present invention, so as to make proliferation of effective microorganisms dominant.

### <Deposited Microorganisms>

Deposited microorganisms described herein are as follows.

Bacillus subtilis C-3102 (FERM BP-1096), Bacillus subtilis NBRC 12195, Bacillus subtilis NBRC 14206, Bacillus licheniformis NBRC 12200, Bacillus subtilis sp spizizenii NBRC 101239, Bacillus amyloliquefaciens NBRC 3022, Bacillus amyloliquefaciens NBRC 3032, Bacillus coagulans NBRC 12583, Bacillus coagulans NBRC 3887, Geobacillus stearothermophilus NBRC 13737, and Aeribacillus pallidus TK6004 (FERM BP-08597).

Bacillus subtilis C-3102 (international accession number FERM BP-1096) and Aeribacillus pallidus TK6004 (international accession number FERM BP-08597) were deposited internationally with International Patent Organism Depositary, National Institute of Advanced Industrial Science and Technology (Tsukuba Central 6, 1-1-1 Higashi, Tsukuba-shi, Ibaraki, Postcode 305-8566, Japan) as of 25 Dec. 1985 and 22 Aug. 2002 respectively under the provisions of Budapest Treaty, and currently stored and administered by Patent Microorganisms Depositary, National Institute of Technology and Evaluation (2-5-8 Kazusakamatari, Kisarazu-shi, Chiba, Postcode 292-0818, Japan).

Bacillus subtilis NBRC 12195, Bacillus subtilis NBRC 14206, Bacillus licheniformis NBRC 12200, Bacillus subtilis sp spizizenii NBRC 101239, Bacillus amyloliquefaciens NBRC 3022, Bacillus amyloliquefaciens NBRC 3032, Bacillus coagulans NBRC 12583, Bacillus coagulans NBRC 3887, and Geobacillus stearothermophilus NBRC 13737 were furnished by Patent Microorganisms Depositary, National Institute of Technology and Evaluation (NBRC) (2-5-8 Kazusakamatari, Kisarazu-shi, Chiba, Postcode 292-0818, Japan).

### EXAMPLES

The present invention will now be described more specifically by way of the following examples, provided that the scope of the present invention be not limited to the examples.

### Example 1

### Preparation of Bacterial Spore Suspension

TS culture media (Difco, Trypticase Soy Broth) were inoculated respectively with 11 strains listed in Table 1 and cultured at 50° C. with respect to Geobacillus stearothermophilus NBRC13737 and Aeribacillus pallidus TK6004 (FERM BP-08597), and at 37° C. with respect to the other 9 strains, until respective colonies were established. Then with respect to each of 11 strains, a loop full of a single colony was scratched by a platinum loop and suspended in 500 μL of sterilized water. In 1000 g of water was dissolved 8.0 g of a nutrient agar culture medium (Difco, Nutrient Broth), followed by high pressure steam sterilization (121° C., 15 min) to prepare a plate medium. The nutrient agar culture medium was smeared with each 100 μL of the suspension and cultured at 37° C. for 3 days.

Then, bacterial cells were suspended using a platinum loop in 500 μL of sterilized water, and the bacterial suspension was subjected to a heat shock at 65° C. for 35 min. Each 100 μL of the heat-shocked bacterial suspension and a not-heat-shocked bacterial suspension were smeared on the TS culture media respectively and cultured at 50° C. or 37° C. for 1 day. As the results of the cultures, colony formation was confirmed also with respect to the heat-shocked bacterial suspensions, to confirm that spores were formed for all of 11 strains. Further, since the bacteria were not stained by Gram staining, it was reconfirmed that spores were formed.

After confirming the spore formation, the bacterial cells were collected by a bacteria spreader, suspended in 10 mL of sterilized distilled water, and then centrifuged (8340×g, 4° C., 15 min). The supernatant was removed and thereafter the bacterial cells were washed twice with 10 mL of sterilized water. After washing, the bacterial cells were suspended in 5 mL of sterilized water, and the suspension was filtrated by sterilized Kimwipes. The filtrate was centrifuged (8340×g, 4° C., 15 min) to remove the supernatant, and the rest was adjusted to the turbidity (at 630 nm) of 1.0 with sterilized water.

Used were 11 strains listed in Table 1.

TABLE 1

| Strain name | Accession number |
|---|---|
| Bacillus subtilis C-3102 | FERM BP-1096 |
| Bacillus licheniformis | NBRC 12200^{T} |
| Bacillus licheniformis | NBRC 12195 |
| Bacillus licheniformis | NBRC 14206 |
| Bacillus amyloliquefaciens | NBRC 3022 |
| Bacillus amyloliquefaciens | NBRC 3032 |
| Bacillus coagulans | NBRC 12583^{T} |
| Bacillus coagulans | NBRC 3887 |
| Bacillus subtilis sp spizizenii | NBRC 101239^{T} |
| Geobacillus stearothermophilus | NBRC 13737 |
| Aeribacillus pallidus TK6004 | FERM BP-08597 |

*T stands for “Type strain”

### (Preparation of Germination Inducer)

In 500 mL of distilled water heated to 70° C. was added 0.3 g of ferulic acid, vanillic acid, or L-alanine. After dissolving with stirring using a stirring rod with a propeller, filtration sterilization (0.45 μm filter) was conducted to prepare each 600 ppm-solution.

### (Confirmation of Germination Induction Effect)

Five hundred (500) μL of each solution and 500 μL of each bacterial spore suspension were mixed. The concentration of ferulic acid, vanillic acid, or L-alanine in each of the mixed suspensions is 300 ppm. Defining the time point of mixing as the reaction initiation (0 min), and continuing shaking (150 rpm) at 37° C., the turbidity (630 nm) was measured at 10, 20, 30, 40, 50, 60, 90, 120, and 180 min after the reaction initiation.

Since the turbidity at 630 nm decreases by germination of spores, it has been known that the turbidity at 630 nm can be used to determine the germination rate.

### (Results)

With respect to Bacillus subtilis C-3102 (FERM BP-1096), Bacillus licheniformis (NBRC 12200, NBRC 12195, and NBRC 14206), Bacillus amyloliquefaciens (NBRC 3022, and NBRC 3032), Bacillus coagulans (NBRC 12583, and NBRC 3887), Geobacillus stearothermophilus (NBRC 13737), and Aeribacillus pallidus TK6004 (FERM BP-08597), by mixing either of ferulic acid and vanillic acid the turbidity decreased, thereby to confirm germination. While with respect to Bacillus subtilis sp spizizenii (NBRC101239), by mixing ferulic acid germination was confirmed.

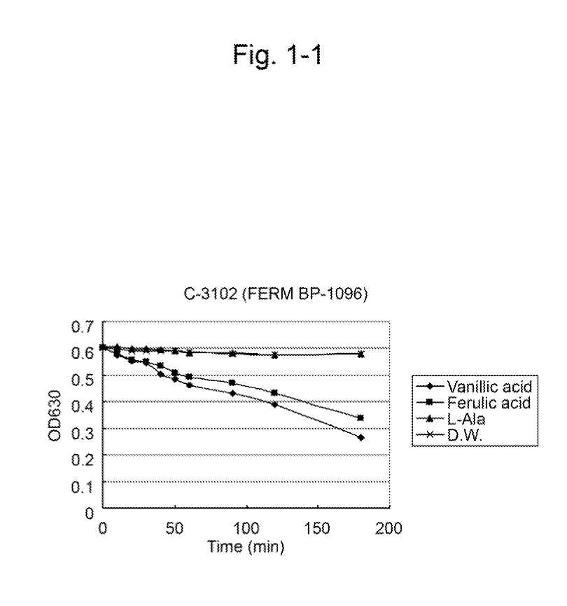

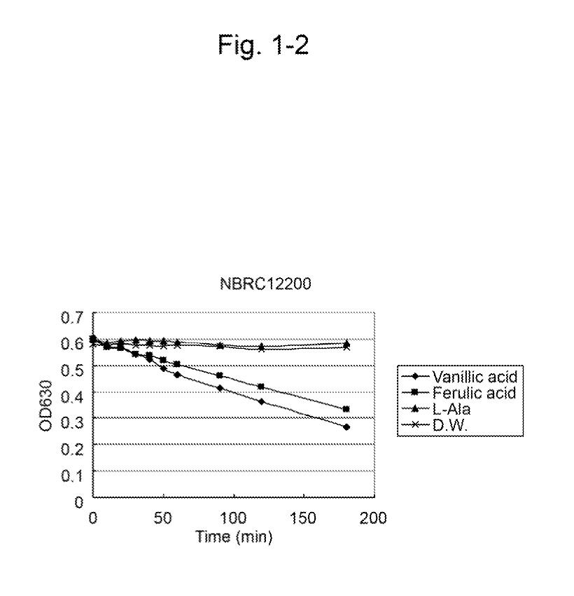

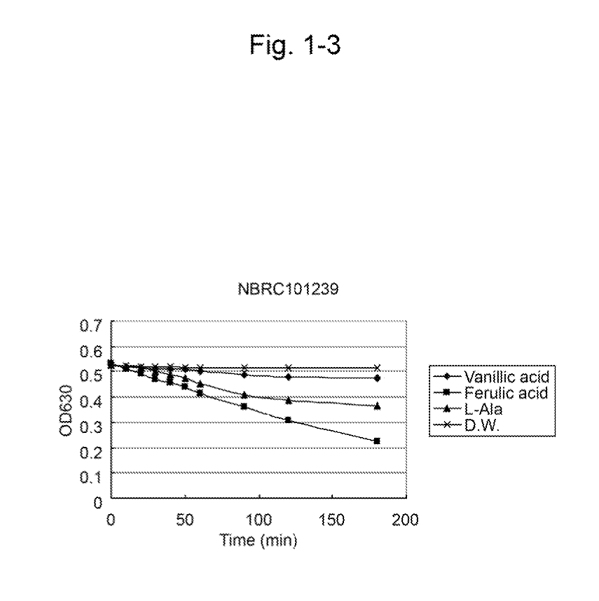

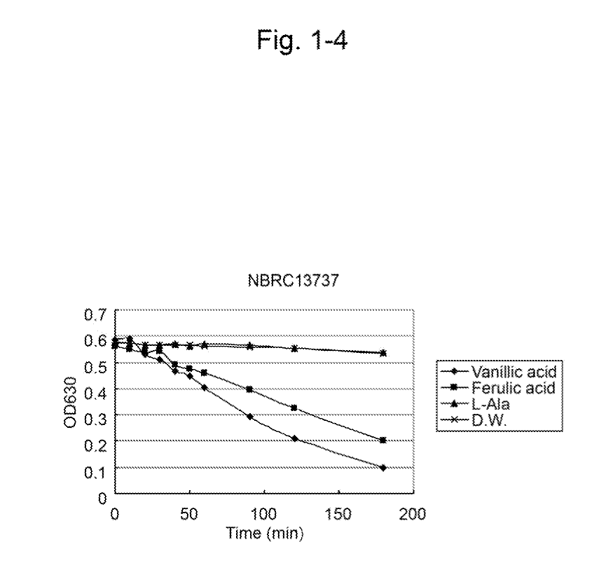

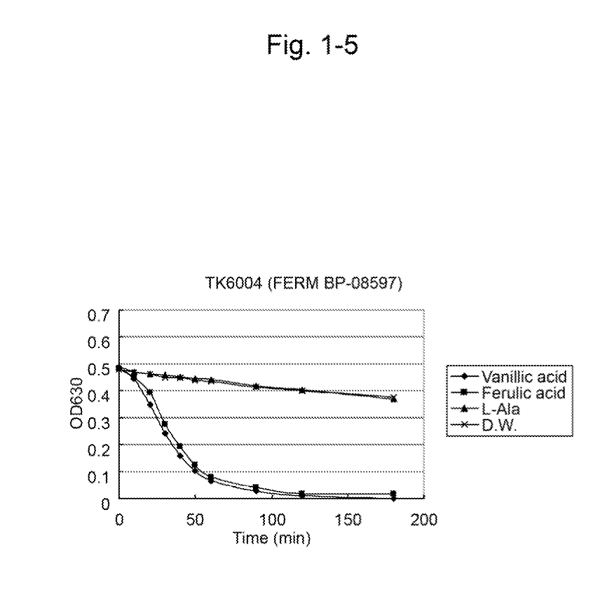

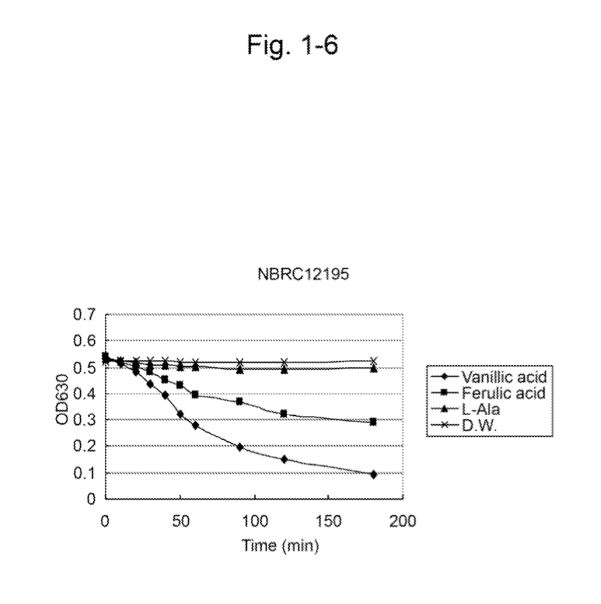

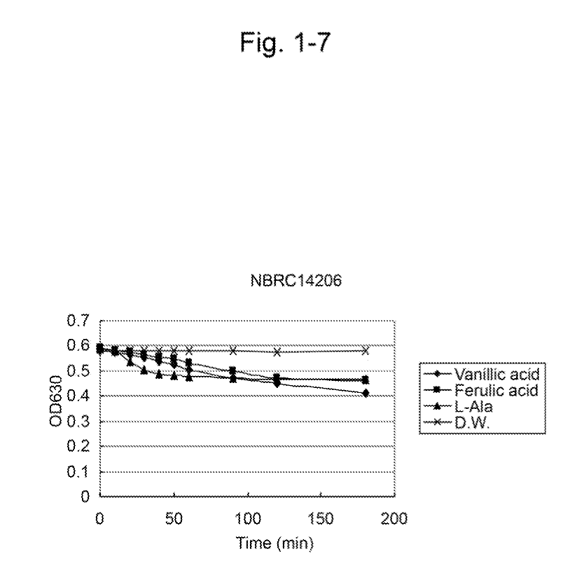

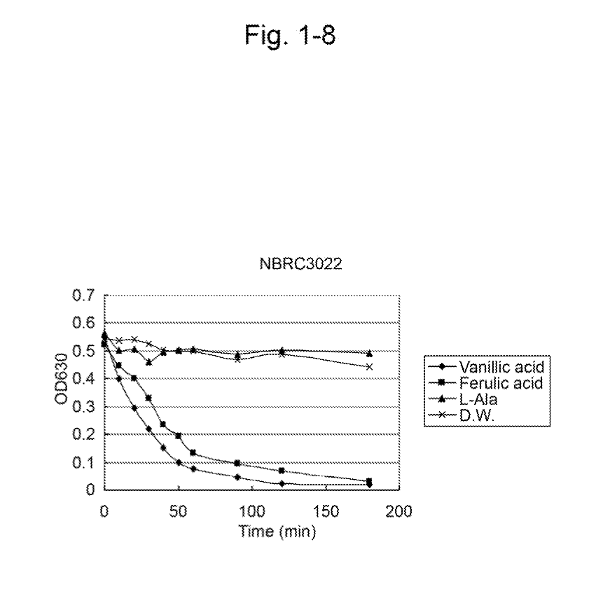

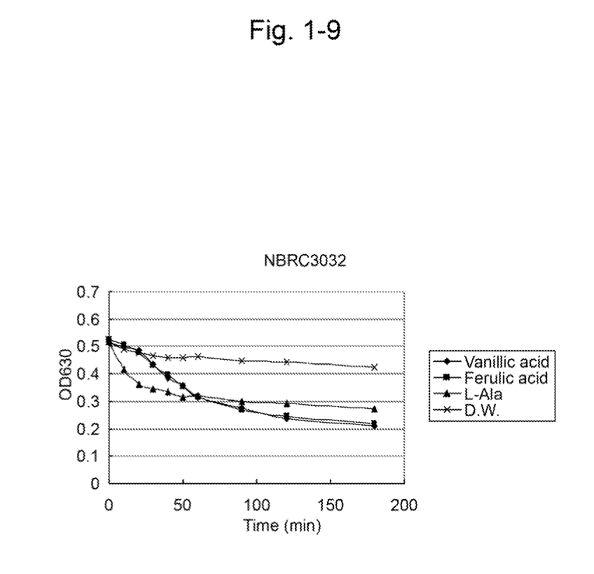

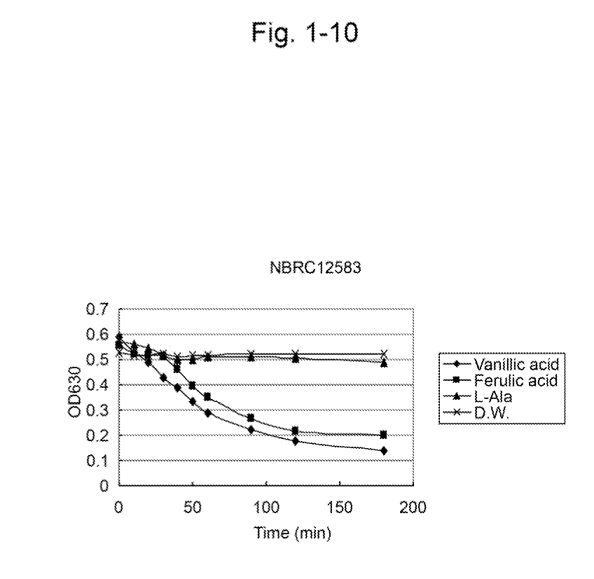

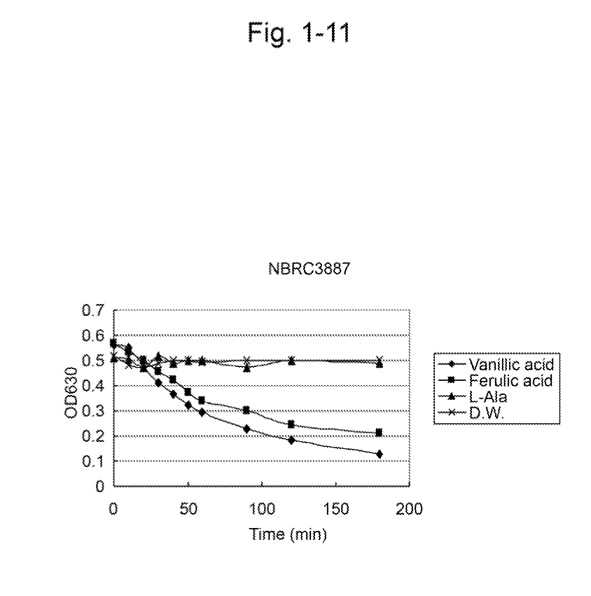

The results of Example 1 are shown in the following Tables 2 to 12 and FIG. 1-1 to FIG. 1-11.

TABLE 2
Change of turbidity (630 nm) after mixture of ferulic acid, vanillic acid,
or L-alanine Bacillus subtilis C-3102 (FERM BP-1096)

| Time after reaction | | | | |
|---|---|---|---|---|
| initiation (min) | Vanillic acid | Ferulic acid | L-Ala | D.W. |
| 0 | 0.604 | 0.602 | 0.606 | 0.603 |
| 10 | 0.575 | 0.578 | 0.607 | 0.596 |
| 20 | 0.551 | 0.558 | 0.597 | 0.589 |
| 30 | 0.543 | 0.548 | 0.597 | 0.592 |
| 40 | 0.505 | 0.535 | 0.594 | 0.592 |
| 50 | 0.483 | 0.506 | 0.590 | 0.591 |
| 60 | 0.461 | 0.493 | 0.582 | 0.585 |
| 90 | 0.432 | 0.471 | 0.584 | 0.579 |
| 120 | 0.390 | 0.430 | 0.574 | 0.577 |
| 180 | 0.266 | 0.335 | 0.578 | 0.579 |

TABLE 3
Change of turbidity (630 nm) after mixture of ferulic acid, vanillic acid,
or L-alanine Bacillus licheniformis (NBRC 12200)

| Time after reaction | | | | |
|---|---|---|---|---|
| initiation (min) | Vanillic acid | Ferulic acid | L-Ala | D.W. |
| 0 | 0.602 | 0.601 | 0.598 | 0.582 |
| 10 | 0.574 | 0.569 | 0.584 | 0.577 |
| 20 | 0.567 | 0.563 | 0.592 | 0.580 |
| 30 | 0.542 | 0.543 | 0.595 | 0.578 |
| 40 | 0.523 | 0.536 | 0.593 | 0.576 |
| 50 | 0.488 | 0.517 | 0.592 | 0.573 |
| 60 | 0.464 | 0.503 | 0.586 | 0.577 |
| 90 | 0.414 | 0.460 | 0.578 | 0.571 |
| 120 | 0.362 | 0.417 | 0.572 | 0.562 |
| 180 | 0.267 | 0.334 | 0.583 | 0.568 |

TABLE 4
Change of turbidity (630 nm) after mixture of ferulic acid, vanillic acid,
or L-alanine Bacillus licheniformis (NBRC 12195)

| Time after reaction | | | | |
|---|---|---|---|---|
| initiation (min) | Vanillic acid | Ferulic acid | L-Ala | D.W. |
| 0 | 0.536 | 0.541 | 0.534 | 0.526 |
| 10 | 0.513 | 0.521 | 0.526 | 0.523 |
| 20 | 0.483 | 0.502 | 0.517 | 0.522 |
| 30 | 0.438 | 0.480 | 0.510 | 0.525 |
| 40 | 0.393 | 0.449 | 0.508 | 0.522 |
| 50 | 0.324 | 0.430 | 0.503 | 0.519 |
| 60 | 0.278 | 0.392 | 0.503 | 0.518 |
| 90 | 0.196 | 0.368 | 0.493 | 0.518 |
| 120 | 0.151 | 0.324 | 0.492 | 0.517 |
| 180 | 0.092 | 0.290 | 0.498 | 0.524 |

TABLE 5
Change of turbidity (630 nm) after mixture of ferulic acid, vanillic acid,
or L-alanine Bacillus licheniformis (NBRC 14206)

| Time after reaction | | | | |
|---|---|---|---|---|
| initiation (min) | Vanillic acid | Ferulic acid | L-Ala | D.W. |
| 0 | 0.583 | 0.589 | 0.591 | 0.588 |
| 10 | 0.573 | 0.583 | 0.580 | 0.582 |
| 20 | 0.563 | 0.573 | 0.535 | 0.582 |
| 30 | 0.553 | 0.565 | 0.502 | 0.580 |
| 40 | 0.537 | 0.553 | 0.490 | 0.581 |
| 50 | 0.526 | 0.547 | 0.482 | 0.578 |
| 60 | 0.503 | 0.530 | 0.477 | 0.579 |
| 90 | 0.470 | 0.498 | 0.470 | 0.579 |
| 120 | 0.453 | 0.474 | 0.465 | 0.577 |
| 180 | 0.414 | 0.463 | 0.468 | 0.579 |

TABLE 6
Change of turbidity (630 nm) after mixture of ferulic acid, vanillic acid,
or L-alanine Bacillus amyloliquefaciens (NBRC 3022)

| Time after reaction | | | | |
|---|---|---|---|---|
| initiation (min) | Vanillic acid | Ferulic acid | L-Ala | D.W. |
| 0 | 0.548 | 0.522 | 0.565 | 0.543 |
| 10 | 0.402 | 0.447 | 0.505 | 0.538 |
| 20 | 0.295 | 0.400 | 0.506 | 0.542 |
| 30 | 0.221 | 0.328 | 0.461 | 0.526 |
| 40 | 0.150 | 0.234 | 0.495 | 0.502 |
| 50 | 0.100 | 0.192 | 0.504 | 0.500 |
| 60 | 0.076 | 0.133 | 0.506 | 0.498 |
| 90 | 0.047 | 0.094 | 0.490 | 0.471 |
| 120 | 0.021 | 0.067 | 0.505 | 0.490 |
| 180 | 0.018 | 0.030 | 0.493 | 0.443 |

TABLE 7
Change of turbidity (630 nm) after mixture of ferulic acid, vanillic acid,
or L-alanine Bacillus amyloliquefaciens (NBRC 3032)

| Time after reaction | | | | |
|---|---|---|---|---|
| initiation (min) | Vanillic acid | Ferulic acid | L-Ala | D.W. |
| 0 | 0.519 | 0.526 | 0.517 | 0.518 |
| 10 | 0.496 | 0.505 | 0.418 | 0.491 |
| 20 | 0.485 | 0.480 | 0.362 | 0.480 |
| 30 | 0.435 | 0.431 | 0.347 | 0.468 |
| 40 | 0.385 | 0.397 | 0.333 | 0.457 |
| 50 | 0.359 | 0.353 | 0.315 | 0.457 |
| 60 | 0.316 | 0.316 | 0.32 | 0.462 |
| 90 | 0.278 | 0.269 | 0.298 | 0.448 |
| 120 | 0.237 | 0.245 | 0.290 | 0.445 |
| 180 | 0.210 | 0.216 | 0.274 | 0.423 |

TABLE 8
Change of turbidity (630 nm) after mixture of ferulic acid, vanillic acid,
or L-alanine Bacillus coagulans (NBRC 12583)

| Time after reaction | | | | |
|---|---|---|---|---|
| initiation (min) | Vanillic acid | Ferulic acid | L-Ala | D.W. |
| 0 | 0.590 | 0.558 | 0.564 | 0.526 |
| 10 | 0.528 | 0.520 | 0.559 | 0.516 |
| 20 | 0.490 | 0.518 | 0.545 | 0.516 |
| 30 | 0.430 | 0.510 | 0.515 | 0.523 |
| 40 | 0.390 | 0.460 | 0.502 | 0.511 |
| 50 | 0.335 | 0.392 | 0.498 | 0.515 |
| 60 | 0.290 | 0.350 | 0.510 | 0.517 |
| 90 | 0.225 | 0.266 | 0.510 | 0.522 |
| 120 | 0.180 | 0.217 | 0.507 | 0.524 |
| 180 | 0.137 | 0.198 | 0.491 | 0.523 |

TABLE 9
Change of turbidity (630 nm) after mixture of ferulic acid, vanillic acid,
or L-alanine Bacillus coagulans (NBRC 3887)

| Time after reaction | | | | |
|---|---|---|---|---|
| initiation (min) | Vanillic acid | Ferulic acid | L-Ala | D.W. |
| 0 | 0.559 | 0.566 | 0.510 | 0.514 |
| 10 | 0.550 | 0.531 | 0.508 | 0.485 |
| 20 | 0.473 | 0.500 | 0.470 | 0.471 |
| 30 | 0.411 | 0.454 | 0.515 | 0.489 |
| 40 | 0.364 | 0.423 | 0.488 | 0.499 |
| 50 | 0.321 | 0.372 | 0.500 | 0.499 |
| 60 | 0.295 | 0.340 | 0.500 | 0.496 |
| 90 | 0.230 | 0.300 | 0.472 | 0.502 |
| 120 | 0.182 | 0.247 | 0.500 | 0.498 |
| 180 | 0.130 | 0.209 | 0.490 | 0.498 |

TABLE 10
Change of turbidity (630 nm) after mixture of ferulic acid, vanillic acid,
or L-alanine Bacillus subtilis sp spizizenii (NBRC 101239)

| Time after reaction | | | | |
|---|---|---|---|---|
| initiation (min) | Vanillic acid | Ferulic acid | L-Ala | D.W. |
| 0 | 0.529 | 0.534 | 0.527 | 0.522 |
| 10 | 0.520 | 0.512 | 0.520 | 0.524 |
| 20 | 0.516 | 0.495 | 0.514 | 0.520 |
| 30 | 0.512 | 0.471 | 0.501 | 0.520 |
| 40 | 0.510 | 0.458 | 0.488 | 0.520 |
| 50 | 0.510 | 0.439 | 0.475 | 0.517 |
| 60 | 0.502 | 0.414 | 0.455 | 0.517 |
| 90 | 0.488 | 0.362 | 0.409 | 0.515 |
| 120 | 0.479 | 0.306 | 0.387 | 0.516 |
| 180 | 0.474 | 0.224 | 0.366 | 0.515 |

TABLE 11
Change of turbidity (630 nm) after mixture of ferulic acid, vanillic acid,
or L-alanine Geobacillus stearothermophilus (NBRC 13737)

| Time after reaction | | | | |
|---|---|---|---|---|
| initiation (min) | Vanillic acid | Ferulic acid | L-Ala | D.W. |
| 0 | 0.584 | 0.560 | 0.577 | 0.572 |
| 10 | 0.590 | 0.551 | 0.573 | 0.572 |
| 20 | 0.531 | 0.537 | 0.566 | 0.566 |
| 30 | 0.510 | 0.541 | 0.565 | 0.566 |
| 40 | 0.467 | 0.489 | 0.568 | 0.566 |
| 50 | 0.445 | 0.473 | 0.561 | 0.564 |
| 60 | 0.402 | 0.459 | 0.568 | 0.561 |
| 90 | 0.291 | 0.397 | 0.565 | 0.557 |
| 120 | 0.209 | 0.326 | 0.553 | 0.555 |
| 180 | 0.099 | 0.202 | 0.538 | 0.535 |

TABLE 12
Change of turbidity (630 nm) after mixture of ferulic acid, vanillic acid,
or L-alanine Aeribacillus pallidus TK6004 (FERM BP-08597)

| Time after reaction | | | | |
|---|---|---|---|---|
| initiation (min) | Vanillic acid | Ferulic acid | L-Ala | D.W. |
| 0 | 0.482 | 0.484 | 0.488 | 0.478 |
| 10 | 0.444 | 0.450 | 0.470 | 0.468 |
| 20 | 0.348 | 0.393 | 0.463 | 0.461 |
| 30 | 0.243 | 0.276 | 0.457 | 0.450 |
| 40 | 0.158 | 0.193 | 0.451 | 0.447 |
| 50 | 0.103 | 0.124 | 0.445 | 0.439 |
| 60 | 0.067 | 0.080 | 0.441 | 0.435 |
| 90 | 0.026 | 0.040 | 0.416 | 0.415 |
| 120 | 0.010 | 0.016 | 0.404 | 0.400 |
| 180 | 0.000 | 0.016 | 0.370 | 0.377 |

### Example 2

### Adjustment of Concentration of Germination Inducer

Ferulic acid 0.01, 0.1, 0.3, and 0.6 g, and vanillic acid 0.01, 0.1, 0.3, 0.6, 1.2, 2.4, and 3.6 g were each mixed with 500 mL of distilled water at 70° C. to prepare 20, 200, 600, and 1200 ppm-ferulic acid solutions, and 20, 200, 600, 1200, 2400, and 3600 ppm-vanillic acid solutions.

Next, 500 μL aliquots of bacterial suspension of Bacillus subtilis C-3102 (FERM BP-1096) used in Example 1 were mixed with 500 μL of ferulic acid, or vanillic acid solutions described above, and the mixtures were adjusted to ferulic acid (10 to 600 ppm), or vanillic acid (10 to 1800 ppm) concentrations set forth in Table 2. The concentrations of ferulic acid at the reaction initiation were 10, 100, 300, and 600 ppm, and the concentrations of vanillic acid at the reaction initiation were 10, 100, 300, 600, 1200, and 1800 ppm.

As same as Example 1, defining the time point of mixing the bacterial suspension with the solution of ferulic acid, or vanillic acid as the reaction initiation (0 min), and continuing shaking (150 rpm) at 37° C., the absorbance (630 nm) was measured at 10, 20, 30, 40, 50, 60, 90, 120, and 180 min after the reaction initiation.

### (Results)

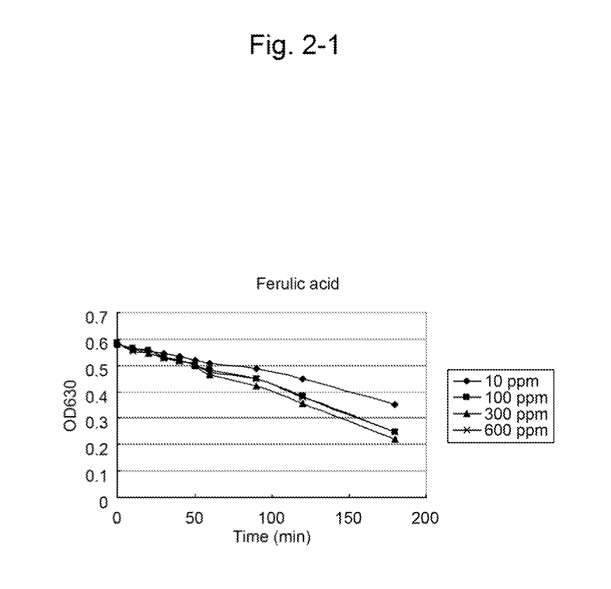

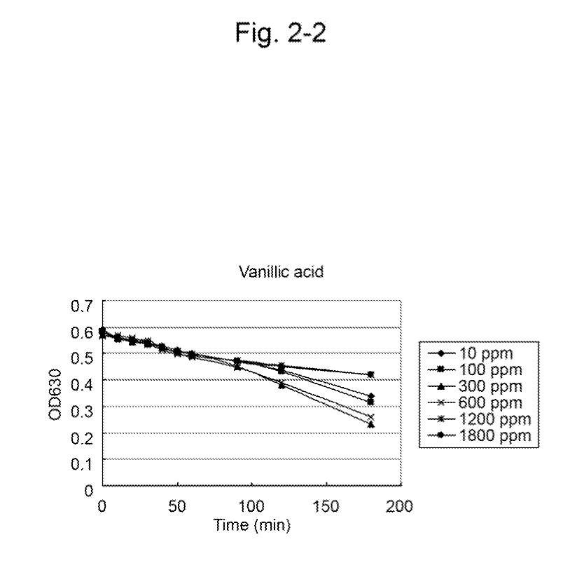

The results of Example 2 are shown in the following Table 13 and Table 14 as well as in FIG. 2-1 and FIG. 2-2. When germination was induced by adding ferulic acid of 10 to 600 ppm, and vanillic acid of 10 to 1800 ppm, it was shown that germination was inducted at any concentration to the same extent in Bacillus subtilis C-3102. From this it is suggested that ferulic acid from 10 ppm to 600 ppm, and vanillic acid from 10 ppm to 1800 ppm have similar germination induction effect on bacteria of the genus Geobacillus and the genus Aeribacillus, as on bacteria of the genus Bacillus.

TABLE 13
Turbidity change with respect to Bacillus subtilis C-3102 by mixing
ferulic acid at various concentrations (10 to 600 ppm)

| | |
|---|---|
| | Concentration of |
| Time after reaction | Ferulic acid (ppm) |

| initiation (min) | 10 | 100 | 300 | 600 |
|---|---|---|---|---|
| 0 | 0.584 | 0.583 | 0.579 | 0.586 |
| 10 | 0.563 | 0.565 | 0.56 | 0.552 |
| 20 | 0.553 | 0.556 | 0.547 | 0.545 |
| 30 | 0.544 | 0.53 | 0.530 | 0.526 |
| 40 | 0.533 | 0.52 | 0.520 | 0.515 |
| 50 | 0.519 | 0.500 | 0.499 | 0.506 |
| 60 | 0.507 | 0.484 | 0.466 | 0.477 |
| 90 | 0.488 | 0.448 | 0.422 | 0.447 |
| 120 | 0.447 | 0.383 | 0.354 | 0.379 |
| 180 | 0.353 | 0.247 | 0.22 | 0.246 |

TABLE 14
Turbidity change with respect to Bacillus subtilis C-3102 by
mixing vanillic acid at various concentrations (10 to 1800 ppm)

| Time after reaction | Concentration of Vanillic acid (ppm) | | | | | |
|---|---|---|---|---|---|---|
| initiation (min) | 10 | 100 | 300 | 600 | 1200 | 1800 |
| 0 | 0.590 | 0.584 | 0.572 | 0.566 | 0.567 | 0.579 |
| 10 | 0.558 | 0.557 | 0.555 | 0.568 | 0.559 | 0.562 |
| 20 | 0.548 | 0.553 | 0.543 | 0.561 | 0.551 | 0.549 |
| 30 | 0.539 | 0.537 | 0.538 | 0.540 | 0.548 | 0.538 |
| 40 | 0.527 | 0.526 | 0.526 | 0.512 | 0.520 | 0.519 |
| 50 | 0.514 | 0.501 | 0.507 | 0.499 | 0.509 | 0.510 |
| 60 | 0.497 | 0.491 | 0.501 | 0.488 | 0.496 | 0.498 |
| 90 | 0.472 | 0.470 | 0.452 | 0.447 | 0.474 | 0.472 |
| 120 | 0.437 | 0.433 | 0.381 | 0.390 | 0.456 | 0.453 |
| 180 | 0.338 | 0.315 | 0.235 | 0.262 | 0.419 | 0.420 |

### Example 3

### Preparation of Germination Inducer

In each 500 mL of distilled water heated to 70° C. was added 0.3 g of ferulic acid, vanillic acid, or p-coumaric acid. After dissolving with stirring using a stirring rod with a propeller, filtration sterilization (0.45 μm filter) was conducted to prepare each 600 ppm-solution.

### (Confirmation of Germination Induction Effect)

Next, 500 μL aliquots of bacterial suspension of Bacillus subtilis C-3102 (FERM BP-1096) prepared by a similar method as in Example 1 were mixed with 500 μL of each of the ferulic acid, vanillic acid, and p-coumaric acid solutions, and the mixtures were adjusted to 300 ppm of ferulic acid, vanillic acid, or p-coumaric acid concentration. Defining the time point of mixing as the reaction initiation (0 min), and continuing shaking (150 rpm) at 37° C., the turbidity (630 nm) was measured at 10, 20, 30, 40, 50, 60, 90, 120, and 180 min after the reaction initiation.

### (Results)

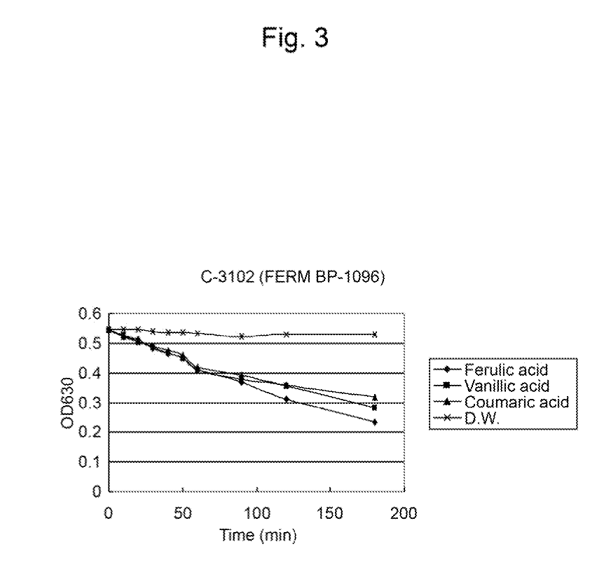

The results of Example 3 are shown in the following Table 15 and in FIG. 3. When germination was induced by adding ferulic acid, vanillic acid, and p-coumaric acid at 300 ppm, the turbidity with respect to Bacillus subtilis C-3102 decreased, thereby to confirm germination. From this it is suggested that a compound having a phenolic backbone, such as ferulic acid, vanillic acid, or p-coumaric acid, has, irrespective of its type, a germination induction effect.

TABLE 15
Turbidity change with respect to Bacillus subtilis C-3102 by mixing
ferulic acid, vanillic acid, or p-coumaric acid at 300 ppm

| Time after reaction | | | p-Coumaric | |
|---|---|---|---|---|
| initiation (min) | Ferulic acid | Vanillic acid | acid | D.W. |
| 0 | 0.545 | 0.542 | 0.545 | 0.546 |
| 10 | 0.527 | 0.525 | 0.523 | 0.547 |
| 20 | 0.513 | 0.506 | 0.506 | 0.545 |
| 30 | 0.484 | 0.485 | 0.488 | 0.538 |
| 40 | 0.466 | 0.467 | 0.475 | 0.536 |
| 50 | 0.449 | 0.450 | 0.460 | 0.535 |
| 60 | 0.413 | 0.409 | 0.420 | 0.532 |
| 90 | 0.368 | 0.379 | 0.392 | 0.524 |
| 120 | 0.313 | 0.356 | 0.360 | 0.528 |
| 180 | 0.233 | 0.282 | 0.319 | 0.528 |

### Example 4

### Verifying that Ferulic Acid or Vanillic Acid is not Utilized as a Nutrient Component in the Method of the Present Invention

This Example is an experiment to examine whether proliferation is recognizable or not, when ferulic acid or vanillic acid is mixed with a spore suspension of the C-3102 strain and cultured for a long time period.

The C-3102 strain was cultured on a nutrient plate at 37° C. for 3 days, and thereafter bacterial cells were collected by a bacteria spreader and a spore suspension was prepared similarly as in Example 1 (OD 630=1.0). After mixing 500 μL of the C-3102 spore suspension with 500 μL of a 600 ppm-ferulic acid solution, or a 600 ppm-vanillic acid solution, change in the turbidity (630 nm) up to 24 hours was examined. The results are shown in Table 16.

TABLE 16

| Reaction time (hr) | Ferulic acid | Vanillic acid | D.W. |
|---|---|---|---|
| 0 | 0.574 | 0.575 | 0.593 |
| 0.5 | 0.543 | 0.540 | 0.582 |
| 1 | 0.502 | 0.481 | 0.575 |
| 1.5 | 0.464 | 0.426 | 0.568 |
| 2 | 0.425 | 0.380 | 0.564 |
| 3 | 0.370 | 0.316 | 0.551 |
| 24 | 0.178 | 0.060 | 0.527 |

As clearly indicated in the table, even after a long time culture with a ferulic acid solution or a vanillic acid solution, increase in the turbidity (i.e., the proliferation of C-3102) was not recognized. As shown in the above Examples, when germination is induced, the turbidity decreases (namely, due to degradation of a spore membrane, the light refraction decreases so that the turbidity decreases). Meanwhile, it is conceivable that, if induced vegetative cells proliferate, the turbidity simply increases. From the results in the table of 24-hour observation of a mixture liquid with ferulic acid or a mixture liquid with vanillic acid there was no increase in the turbidity recognized, which indicates that ferulic acid and vanillic acid have spore induction effect, but are singly incompetent as a nutrient component.

Considering the above results, although in Non Patent Literature 1 (G. Gurujeyalakshmi and A. Mahadevan, Current Microbiology, 1987; 16: 69-73) there is a description about a bacterial population increases in a culture medium containing ferulic acid, because of absence of a sporulating treatment, the literature does not indicates a germination induction effect.

### INDUSTRIAL APPLICABILITY

The present invention can induce and promote germination of spore forming bacteria, such as bacteria of the genus Bacillus useful in the agricultural field, so as to increase the proliferation rate upon contacting a nutrient component, and to intensify their intrinsic bacterial function or activity; and also make disinfection of such bacteria easier through promoting germination of a spore forming bacterium, which causes deterioration of food.

All the publications, patents, and patent applications cited herein are incorporated by reference herein in their entirety.

1. A method for inducing germination of a spore forming bacterium, comprising: using one or more compounds having a phenolic backbone to induce germination of a spore forming bacterium selected from the group consisting of the genus Bacillus, the genus Geobacillus, and the genus Aeribacillus.

2. The method according to claim 1, wherein the compound is vanillin, vanillic acid, ferulic acid, or coumaric acid, or a salt thereof.

3. The method according to claim 1, wherein the spore forming bacterium is Bacillus subtilis, Bacillus licheniformis, Bacillus amyloliquefaciens, or Bacillus coagulans, Geobacillus stearothermophilus, or Aeribacillus pallidus.

4. The method according to claim 1, wherein the effective concentration of the compound is 1 to 4000 ppm.

5. A germination inducer, which is for a spore forming bacterium belonging to the genus Bacillus, the genus Geobacillus, or the genus Aeribacillus, comprising one or more compounds having a phenolic backbone.

6. The germination inducer according to claim 5, wherein the compound is vanillin, vanillic acid, ferulic acid, or coumaric acid, or a salt thereof.

7. The germination inducer according to claim 5, wherein the spore forming bacterium is Bacillus subtilis, Bacillus lichenifbrmis, Bacillus amyloliquefaciens, or Bacillus coagulans, Geobacillus stearothermophilus, or Aeribacillus pallidus.

8. A kit for use in germination of a spore forming bacterium, comprising: a bacterial cell selected from the group consisting of the genus Bacillus, the genus Geobacillus, and the genus Aeribacillus, wherein the bacterial cell have formed a spore, and/or a spore thereof; and the germination inducer according to any one of claims 5 to 7.

9. The kit for germination according to claim 8, wherein the spore forming bacterium is Bacillus subtilis, Bacillus licheniformis, Bacillus amyloliquefaciens, Bacillus coagulans, Geobacillus stearothermophilus, or Aeribacillus pallidus.

10. The kit for germination according to claim 8, wherein the spore forming bacterium is Bacillus subtilis C-3102 (FERM BP-1096), Bacillus subtilis NBRC 12195, Bacillus subtilis NBRC 14206, Bacillus licheniformis NBRC 12200, Bacillus subtilis sp spizizenii NBRC 101239, Bacillus amyloliquefaciens NBRC 3022, Bacillus amyloliquefaciens NBRC 3032, Bacillus coagulans NBRC 12583, Bacillus coagulans NBRC 3887, Geobacillus stearothermophilus NBRC 13737, or Aeribacillus pallidus TK 6004 (FERM BP-08597).
